(19) United States
(12) Patent Application Publication
(10) Pub. No.: US 2024/0417294 A1
(43) Pub. Date: Dec. 19, 2024

(54) OPTIMISED BIO-ELECTROCHEMICAL REACTOR, IN PARTICULAR FOR DEGRADATION OF THE CHEMICAL OXYGEN DEMAND OF AN EFFLUENT

(71) Applicants: SUEZ INTERNATIONAL, Paris La Défense Cedex (FR); INSTITUT NATIONAL DE RECHERCHE POUR L'AGRICULTURE L'ALIMENTATION ET L'ENVIRONNEMENT, PARIS (FR)

(72) Inventors: Roman MOSCOVIZ, Mauremont (FR); Théodore BOUCHEZ, Villemoisson (FR); Florence DE FOUCHÉCOUR, Jouy-en-Josas (FR); Yannick FAYOLLE, Paris (FR)

(73) Assignees: SUEZ INTERNATIONAL, Paris La Défense Cedex (FR); INSTITUT NATIONAL DE RECHERCHE POUR L'AGRICULTURE L'ALIMENTATION ET L'ENVIRONNEMENT, PARIS (FR)

(21) Appl. No.: 18/563,491

(22) PCT Filed: May 20, 2022
(86) PCT No.: PCT/FR2022/050969

§ 371 Date: Nov. 22, 2023

(51) Int. Cl. C02F 3/00; C02F 1/461; C12M 1/12; C25B 1/02; C25B 3/03; C25B 3/07; C25B 3/26; C25B 9/13; H01M 8/16

(52) U.S. Cl. CPC C02F 3/005; C02F 1/461; C12M 25/16; C25B 1/02; C25B 3/03; C25B 3/07; C25B 3/26; C25B 9/13; H01M 8/16

(57) ABSTRACT

An optimised bio-electrochemical reactor for treating a liquid effluent containing a biodegradable organic pollution. At least one of the anode and cathode compartments of the reactor is a microbial biofilm compartment (12) including a multi-stage current collector immersed in an electrolyte comprising electroactive microorganisms. This current collector includes at least two stages, each defining a chamber acting as a container for a biocompatible granular support material and letting the fluid pass through. The effluent circulates inside the compartment (12) according to an X direction crossing the stages of the current collector. In operation, the support material (17) is in a fluidised state resulting either from the circulation of the effluent, or from the circulation of a fluidisation gas allowing optimising the active surface of the electrode and, consequently, the treatment of the effluent.

### FIELD OF THE INVENTION

The innovation relates to the field of treatment and valorization of the effluents containing a biodegradable organic pollution, such as wastewater (municipal or industrial) or organic waste such as manure or leachate, and more particularly to electrochemical systems and methods implementing bio-electrochemical reactors, i.e. electrochemical devices wherein at least one of the electrodes of which, so-called bio-electrode, is in contact with microorganisms.

### TECHNOLOGICAL BACKGROUND

Such electrochemical systems and methods, which are relatively recent technologies (about 20 years), enable in particular coupling of the treatment of effluents containing organic matter (wastewater, hydrolysate, etc.) with the production of electrical energy or molecules with added value, such as organic acids and/or alcohols. In the first case, we talk about microbial fuel cells, whereas in the second case these consist of microbial electrolysers. In both cases, at least one of the two oxidation or reduction reactions is catalysed by microorganisms, generally in the form of a biofilm. The use of microorganisms generates constraints such as the instability of the biological activity over time, the ageing of the biofilm related to the accumulation of biomass, a sensitivity to some inhibitors, including dioxygen (O₂) in some cases.

Historically, the first configurations of bio-electrochemical systems have been inspired by purely electrochemical technologies of the “stack” type containing planar electrodes arranged parallel to each other. Such a configuration allows minimising energy losses by reducing internal resistances but is barely suited to reactors having large working volumes and makes maintenance of the reactors difficult. In addition, the electrode surface/volume ratios of the anode compartment are generally low, thereby limiting the prospects for intensifying the process or its use for highly diluted effluents.

Thus, these first configurations have been the subject of research in order to develop solutions suited to the treatment of wastewater. A first solution consists in using electrodes in the form of a brush facilitating scaling up, the establishment of a biofilm as well as the maintenance of the bio-electrochemical system. In theory, these brush-shaped electrodes develop a larger useful area than planar electrodes. However, this advantage is rapidly counterbalanced when the brushes are clogged by microbial biofilms. Hence, this configuration also suffers from low electrode/volume ratios of the anode compartment.

Another family of solutions is inspired by processes for treating wastewater by fixed or granular biomass. These solutions use fixed-bed granular electrodes, the electrodes forming compartments are entirely filled with the granular material, as described for example by Zhou, Y. et al. [6], or partially filled with the granular material, as described for example by Jia, Y. H. et al. [2]. These processes aim to optimise the treatment of wastewater: they can treat large volumes because they have a large electrode surface/volume ratio of the anode compartment, but they are not necessarily adapted to electrochemical stresses. In particular, these configurations may impose considerable constraints on the design of the current collector and the geometries of the reactor, and are generally characterised by large internal resistances, leading to significant energy losses.

Thus, for example, Jia, Y. H. et al. [2] have described a microbial electrolysis cell allowing producing hydrogen gas from biomass by a process catalysed by microorganisms. The used bioreactor comprises an anode compartment and a cathode compartment separated by a membrane, the anode compartment comprising modules forming metal baskets filled with graphite granules on which the microorganisms are deposited. These modules are arranged vertically, horizontally side-by-side, parallel to each other with a spacing. Thus, the flow of the effluent to be treated is preferably done along the modules, and not through it, which does not promote exchanges with the graphite granules and the microorganisms. In particular, the granules located inside the baskets cannot be fluidised.

Other solutions using fluidised-bed granular electrodes have been developed. Thus, Li, J. et al. [3] have studied a membrane bio-electrochemical reactor (also so-called “MBER”, standing for “Membrane Bio-electrochemical Reactor”) using a fluidised bed in order to reduce clogging of the membrane. The reactor is formed of a cation-exchange membrane with a tubular shape. The anode is a carbon fabric supported by a metal mesh arranged inside the tubular membrane, along the inner wall of the latter. The interior of the tubular membrane comprises hollow membranes in the form of hollow fibres and activated carbon particles. The cathode is formed of a carbon fabric covered with Pt/C powder surrounding the tubular membrane. Finally, microorganisms are introduced into the tubular membrane. In operation, the activated carbon particles located inside the tubular membrane are fluidised. Nonetheless, this study shows that the fluidised-bed reactor MBER cannot be used alone for the treatment of industrial water. It is used as a post-treatment of an industrial water, taking place after a treatment by a microbial fuel cell (also called “MFC”, standing for “Microbial fuel cell”). Liu J. et al. [4] have described a fluidised granular electrode microbial electrolysis cell operating in a batch mode. The reactor is in the form of a tube made of PVC (polyvinyl chloride) comprising activated carbon particles as a fluidised anode, these particles will serve as a support for the microorganisms. A nylon filter positioned at the bottom of the reactor allows avoiding particles coming out of the reactor from below. The anode current collector is formed of a graphite block positioned inside the reactor in its lower half. The cathode is formed of a cylindrical metal mesh positioned at the upper portion of the reactor. A recirculation loop connects the outlet of the reactor to an inlet. In operation, the effluent circulates from the bottom to the top and fluidises the activated carbon particles which will be discharged onto the anode current collector. Nonetheless, during fluidisation, these particles could be entrained up to the cathode, which would disturb the operation of the process. Furthermore, operation in a batch mode is not adapted to an industrial scale.

The article by Liu, J. et al. [5] describes a microbial electrolysis cell comprising two chambers arranged on top of one another and separated by a mesh made of titanium acting as an anode current collector. The lower chamber forms an anode chamber which contains activated carbon granules intended to receive the microorganisms, the upper chamber forms a cathode chamber. In operation, a magnetic stirrer allows fluidising the activated carbon granules inside the anode chamber. This system also operates in a batch mode, which is barely suitable for an industrial scale. Furthermore, positioning one or more membrane(s) between the cathode and anode compartments of the described configuration seems to be unlikely to be considered: an ascending (or descending) vertical hydraulic flow would impose large mechanical stresses on the membrane(s), likely to cause breakup thereof.

Finally, Deeke, A. et al. [1] have described a microbial electrolysis cell comprising a glass column and a distinct discharge cell. The discharge cell comprises the anode and the cathode. The effluent to be treated circulates from the bottom to the top in the glass-made column. Activated carbon particles are introduced into the glass-made column and fluidised by a gaseous nitrogen stream. Thus, they are transported up to the discharge cell and are then transported again in the glass-made column. Nonetheless, this configuration is complex to implement and is barely suitable for an industrial scale. In particular, the particles can be discharged only into the discharge cell, which might limit their discharge frequency. Furthermore, the circulation of the particles between the two portions of the cell requires the use of conduits prone to be clogged, in particular when the granule charge is high.

In general, the systems of the prior art aim to maximise the faradic yield and/or the production of molecules of interest (such as acetate, lactate, etc., biorefinery).

The present invention pursues a different objective: it proposes a bio-electrochemical system that optimises the degradation of organic molecules, in particular for applications on the industrial level.

Hence, the invention aims to overcome the drawbacks of the prior art by providing a bio-electrochemical reactor—in particular an electrolysis or microbial electrosynthesis reactor—capable of treating an effluent containing organic pollution (biodegradable organic matter), such as a wastewater effluent, adaptable to the industrial scale.

The invention also aims to provide a method for treating or pre-treating an effluent containing in particular organic pollution (biodegradable organic matter) comprising a bio-electrochemical reaction step.

The system and the method of the invention are energy-efficient, and competitive compared to a treatment using an aeration basin both in terms of reduction of the COD (Chemical Oxygen Demand) and of energy consumption.

### DESCRIPTION OF THE INVENTION

To this end, the present invention relates to a bio-electrochemical reactor for treating a liquid effluent, comprising at least one anode compartment and at least one cathode compartment.

According to the invention, at least one amongst the anode and cathode compartments (i.e. at least one compartment selected from among the at least one anode compartment and the at least one cathode compartment or both compartments) is a microbial biofilm compartment including:
at least one fluid inlet located at one end of the compartment,
at least one fluid outlet located at an opposite end of the compartment,
means for circulating the fluid inside the compartment between the at least one inlet and the at least one outlet according to an X direction,
an electrolyte comprising electroactive microorganisms,
a biocompatible granular support material,
a multi-stage current collector located between the at least one fluid inlet and the at least one fluid outlet of said microbial biofilm compartment, said collector comprising at least two stages each defining a chamber serving as a container for the granular support material and letting the fluid pass.

In addition, the direction X of circulation of the fluid inside the compartment is not parallel, and preferably orthogonal, or substantially orthogonal, to a Z direction extending from the anode compartment to the cathode compartment. Advantageously, this X direction may be parallel to the direction of gravity to facilitate fluidisation. The chambers of the current collector are then positioned on top of one another. As shown in the figures described later on with reference to examples of implementation of the present invention, the current collector conforms to the shape of the microbial biofilm compartment over the height of said collector measured according to the X direction. Henceforth, each chamber of a stage of the current collector has a section that extends over the entire surface of an inner section of the microbial biofilm compartment, these sections being defined in a plane perpendicular to the X direction. In other words, each chamber conforms to the shape of the microbial biofilm compartment. Thus, when the effluent to be treated circulates inside the microbial biofilm compartment, it passes through the granular support material of each chamber of a stage of the current collector over the entire surface of the inner section of the microbial biofilm compartment.

Finally, inside at least one chamber of the current collector, so-called the fluidisation chamber, a height at rest of the granular support material measured according to the X direction in the absence of fluid circulation inside the microbial biofilm compartment, is adapted to a fluidisation of said granular support material. This height at rest is smaller than the total height of the chamber. Thus, the circulation of the effluent to be treated at an appropriate flow rate throughout the chambers located inside the microbial biofilm compartment, possibly supplemented by the circulation of a fluidisation gas introduced to this end into the reactor, allows fluidising the granular support material. When present, the fluidisation gas is introduced into the reactor via at least one dedicated inlet connected to a conduit provided with means for circulating the fluidisation gas. Alternatively, the fluidisation gas may be mixed with the fluid to be treated before introduction thereof into the reactor.

This fluidisation is obtained by the circulation of the effluent to be treated (possibly supplemented by the circulation of a fluidisation gas) by the circulation means with a flow rate higher than or equal to a minimum flow rate of fluidisation of the granular support material contained inside the current collector. In other words, the circulation 35 means consist of means for circulating at a flow rate higher than or equal to a minimum fluidisation flow rate the granular support material contained inside the current collector.

This configuration in which the microbial film compartment(s) comprises(comprise) one or more fluidised material stage(s) (when the effluent circulates) brings in a good trade-off between the capacity and quality of treatment of the reactor and its electrochemical performances.

In particular, this particular arrangement allows improving the contact between the electroactive microorganisms by maximising the attachment surface for the microorganisms, in particular with respect to systems equipped with non-fluidised granular electrodes. Thus, it is possible to improve the treatment of the effluent.

Furthermore, the multi-stage structure of the current collector allows facilitating and homogenising the fluidisation of the granular support material at each fluidised stage. Thus, it is possible to obtain a relatively homogeneous fluidisation over all of the fluidised stages, in other words over the entire volume of the compartment occupied by these stages and in particular over the entire height and/or volume of the current collector when the stages are distributed over the entire height and/or volume of the latter. Thus, the dead volumes in the microbial biofilm compartment and clogging are minimised, also improving the treatment of the effluent.

Moreover, the fluidisation takes place according to an X direction which is not parallel, and preferably orthogonal to a direction connecting the anode and cathode compartments. This feature, added to the relatively homogeneous fluidisation over the entire height and/or the volume of the compartment occupied by the fluidisation stages, allows in particular stabilising the current lines in the reactor.

By friction effects, the fluidisation also allows moderating the formation of the biofilm, and therefore the sludge generated during operation of the reactor. Thus, the reactor and the method of the invention allow limiting the size of the biological aggregates produced during the treatment of the effluent, which limits the accumulation of the matters in the reactor and forms a major advantage in a process for treating an effluent. The moderation of the formation of the biofilm also allows taking the best advantage of the potential capacitive properties of the granular support material. To sum up, fluidisation allows limiting the apparent growth yields of the microorganisms yet without altering their activity.

The multi-stage structure of the current collector also allows for a great flexibility in the operation and maintenance of the reactor. Indeed, it is possible to replace the granular support material of one stage independently of the other stage(s). we may then talk about a differential renewal of the granular material for each stage. In particular, the current collector may be formed of a structure in one single portion, each chamber being for example equipped with a door for loading the support material, or else, the current collector could be formed of a structure in several portions, for example in several modules electrically connected to each other, each module defining a chamber equipped with a door or having an upper opening open to the material of a basket. For easy handling, the current collector or each module of the current collector may be removable.

It is also possible to use support materials and/or consortia of different microorganisms for each stage. This allows adapting the support material (material, size, number, density, etc.) and/or the populations of microorganisms according to the pursued objectives. This flexibility allows considering the specialisation of the stages in a particular treatment. In particular, it is possible to provide for specialised populations of microorganisms in aerobic treatments for the stage the closest to the effluent inlet, the microorganisms taking advantage of the traces of oxygen dissolved in the effluent (for example specialised for the oxidation of the COD by O₂ dissolved in the effluent). In turn, the next stages would be specialised in anaerobic treatments. The aerobic “chamber” may also be used to protect downstream chambers which must be strictly anaerobic for a proper development of the electroactive microorganisms.

Thus, the electroactive microorganisms may consist of aerobic or anaerobic microorganisms. These microorganisms differ depending on the electrode on which they develop in the form of a biofilm, and the characteristics of the electrolyte in which they are immersed. For example, when wastewater or biowaste hydrolysates are injected into an anode electrolyte, an abundant population affiliated with the genus Geobacter is observed. On the other hand, in a saline medium, other genera such as Geoalkalibacter or Desulforomonas may become dominant. Thus, when the microorganisms are located on the anode, we talk about anodic electroactive microorganisms, whereas when the microorganisms are located on the cathode, we talk about cathodic or electrotrophic electro-active microorganisms.

Advantageously, the at least one microbial biofilm compartment may include one or more circuit(s) for recycling the treated effluent. Thus, this compartment may comprise at least one recycling circuit connecting the at least one outlet to the at least one fluid inlet or connecting the at least one outlet to at least one recycling inlet opening into one of the chambers or upstream one of the chambers with respect to the circulation of the fluid. This may allow facilitating the fluidisation of the support material and increasing the stay time of the effluent in the compartment. When the treated effluent (coming out of the microbial biofilm compartment) is returned to one of the fluidisation chambers, it is possible to perform this return to a chamber other than that or those receiving the effluent to be treated at first. In other words, the recycled treated effluent may be returned to a stage located downstream (with respect to the circulation of the effluent) of at least one first stage in which the effluent to be treated enters. This may be particularly advantageous, in particular in addition to a specialisation of the different chambers. For example, It is thus possible to specialise the microorganisms of a first chamber of the current collector (from upstream to downstream with respect to the circulation of the effluent) in the aerobic degradation of the incoming COD with the dissolved O₂ contained in the effluent. In this case, recycling of the treated effluent downstream of this first chamber allows avoiding fluidising this first chamber which then serves as a fixed-bed bio-filter and avoiding the transport of dissolved O₂ to the fluidisation chambers located downstream, which may then operate under anaerobic conditions. Thus, it should be understood that it is possible to fluidise one or more of the chambers of the current collector depending on the reactions that one wish to promote.

Advantageously, in general, the at least one microbial biofilm compartment of the reactor according to the invention may include means for introducing a fluidisation gas, for example located upstream of a fluidisation chamber, in particular upstream of the fluidisation chamber the farthest upstream with respect to the circulation of the fluid, for example at one of the ends of the compartment, when the fluidisation gas and the effluent to be treated circulate in co-current. This introduction may then be carried out downstream of at least one chamber of the non-fluidised current collector, the introduction of such a gas, circulating in co-current or in counter-current with respect to the effluent to be treated, may facilitate fluidisation of the granular support material within the fluidisation chamber(s).

The anode compartment may be a microbial biofilm compartment, and, optionally, the cathode compartment may be a microbial biofilm compartment.

Depending on the desired use, the bio-electrochemical reactor may be:
a reactor wherein only the anode compartment of which is a microbial biofilm compartment, for example used, in particular in a bio-anode/abiotic cathode configuration, to couple the treatment of the organic matter of an effluent into the anode compartment (by bio-electrochemical oxidation of the COD), either with the production of H₂ from H₂O at the cathode in the case of a microbial electrolysis process, or with a reduction of O₂ at the cathode for a microbial fuel cell,
a reactor wherein only the cathode compartment of which is a microbial biofilm, for example used in an abiotic anode/bio-cathode configuration, to couple, in the context of an electrolysis, a biologically-catalysed reduction (for example denitrification of an effluent or electrosynthesis reaction) in the cathode compartment with an oxidation of H₂O in the anode compartment,
or a reactor wherein two compartments of which are microbial biofilm compartments. In operation as a microbial electrolyser, this reactor type may be used in bio-electrosynthesis processes coupling a treatment of the effluent at the anode and the synthesis of carbon molecules at the cathode. In operation as a microbial fuel cell, this reactor type may be used to couple a treatment of the effluent at the anode with a denitrification treatment at the cathode of the same effluent or of another effluent.

Thus, by “microbial biofilm compartment”, it should be understood a compartment the electrode of which is catalysed by microorganisms, in other words the electrode of which is a bio-electrode immersed in an electrolyte including electro-active microorganisms. The bio-electrode is herein a granular electrode. In the present invention, when mention is made of a microbial biofilm compartment, the latter is preferably as described in the present invention and includes in particular a multi-stage current collector as described in the present invention.

In the context of the invention, a “bio-electrode” (“bio-anode” or “bio-cathode”) is an electrode covered at least partially with a bacterial biofilm comprising electroactive organisms, i.e. covered at least over a portion of its surface immersed in the electrolyte by a bacterial biofilm. According to one embodiment, all of the immersed surface of the bio-electrode is covered with a biofilm. Alternatively, according to another embodiment, only one portion of the surface of the bio-electrode is covered with a biofilm. In this latter embodiment, the surface covered with a biofilm is sufficient to generate the desired activity, in particular in the case of an oxidation of organic waste hydrolysates or of a bio-electrochemical synthesis.

A bio-electrode may be conditioned by introducing an inoculum into the electrolyte or else by enriching the effluent to be treated with microorganisms of interest. In the context of the invention, the microorganisms of interest are microorganisms responsible for bio-electrosynthesis, bio-cathodic denitrification or bio-electrochemical oxidation of the COD. For example, they comprise bacteria capable of using electrons or hydrogen generated at the cathode to synthesise the desired compounds (such as organic acids or alcohols).

As example, for use in bio-electrosynthesis of compounds such as organic acids or alcohol, the cathode compartment of the reactor of the invention being a microbial biofilm compartment, the inoculum may be prepared from an anaerobic digester sludge, possibly having undergone a pretreatment intended to inactivate the methanogenic microorganisms. Thus, this digester sludge may undergo a heat treatment at a temperature and for a time long enough to inactivate the methanogenic microorganisms. The pretreatment may also comprise the enrichment of the waste into microorganisms of interest. In particular, this step may comprise the addition of hydrogen and carbon dioxide, for example in a closed flask in a batch mode. The culture resulting from this enrichment may be used directly and be introduced into the cathode compartment upon startup of the reactor.

In one embodiment, the reactor according to the invention may be used as a microbial fuel cell to produce electric current.

In another embodiment, the reactor may be an electrolysis reactor or a microbial electrosynthesis reactor comprising means for applying a potential difference between the current collector of the microbial biofilm compartment and the electrode of the other compartment. It can then be used to produce dihydrogen (H₂) or chemical molecules of interest (methane, organic acids, alcohol, etc.).

In particular, when the reactor is a microbial electrosynthesis reactor, the microbial biofilm cathode compartment may include at least one other inlet for a carbon source, typically injected in the form of a gas, such as CO₂, biogas, or syngas, and/or introduced in solution in the form of organic carbon: for example acetate and/or in the form of mineral carbon, in particular a bicarbonate.

The bio-electrochemical reactor according to the invention may comprise at least one separator located between the at least one anode compartment and the at least one cathode compartment, for example when soluble molecules are produced, typically at the cathode.

In general, the separator enables the passage of the ions (anions or cations) between the anode and cathode compartments. It may comprise one or more ion-exchange membrane(s), a porous ceramic material enabling the passage of ions, or other. In particular, a person skilled in the art may select the separator according to the implemented electrochemical reaction. In the case where the separator has two ion-exchange membranes, it may further comprise an inter-membrane compartment. This type of separator may be used for any application of the bio-electrochemical reactor of the present invention, but according to the uses, other types of separators may be used.

In the case where the anode compartment is a microbial biofilm compartment, the cathode may advantageously have an active surface larger than the total active surface of the bio-anode. This allows stabilising the operation of the reactor, as described in the document W02020/053529. By active surface of a bio-electrode (herein bio-anode or bio-cathode), it should be understood the surface exposed to the electrolyte, this surface being polarised. In the case where the cathode is a bio-cathode, in other words in the case where the reactor also includes a microbial biofilm cathode compartment, the bio-cathode has a greater inertia because of an active surface area larger than the total active surface area of the bio-anode, which allows guaranteeing a particularly stable cathode potential. Indeed, in operation, once the cathode has reached its working potential, the high stability of the potential of the cathode allows in practice better controlling the anodic potential by varying the potential difference between the bio-cathode and the bio-anodes, and without having to resort to a reference electrode. Thus, such a system enables a fine control of the anodic potential and therefore the optimisation of the activity of the anodic biofilm.

Irrespective of the embodiment, the separator may comprise a cation-exchange membrane and an anion-exchange membrane separated from each other by an inter-membrane compartment comprising a device for drawing molecules synthesised within said reactor. Thus, the inter-membrane compartment is able to collect the ions or molecules produced in the anode and/or cathode compartments. For example, the molecules recovered at this inter-membrane compartment may consist of ammonium salts, phosphate salts or others. In general, molecules (typically soluble molecules) are recovered at this compartment when the reactor is an electrosynthesis reactor for the synthesis of molecules of interest.

In the case where such an inter-membrane compartment is present, the membranes may be positioned so that the anode compartment is separated from the cathode compartment, going from the anode compartment to the cathode compartment, by said cation-exchange membrane and said anion-exchange membrane. This is the case of a synthesis of carboxylic acids at the cathode, wherein cations (for example NH₄⁺) of the anode compartment and anions (for example carboxylate ions) of the cathode compartment are recovered. However, it should be noted that the direction of the anion/cation-exchange membranes depends on the molecules to be recovered and may be reversed.

To fill its functions, the current collector is advantageously:
an electrical conductor, and
its structure enables the passage of the fluid to be treated but not that of the granular support material.

Thus, the current collector could be made of, or contain, a carbon-based material, such as graphite, a carbon fibre fabric, etc., or a conductive metal or metal alloy, most often stainless steel, or any other material commonly used to make a current collector.

Advantageously, the current collector may thus have a structure having a multitude of orifices which do not let the particulate material (namely the granular support material) pass through, preferably formed from a perforated plate, a fabric or a mesh. This type of structure has the advantage of not shielding the current lines. Hence, the dimensions of the orifices will be determined by the dimensions of the particles of the granular support material. This structure, in particular when it is formed of a low-rigidity material, such as a fabric, may be reinforced by a support, for example made of stainless steel, to which the structure is secured.

Advantageously, in order to minimise dead volumes, the current collector of the microbial biofilm compartment of the reactor according to the invention may extend over 90 to 100% of the height of the microbial biofilm compartment, this height of the microbial biofilm compartment being defined as the distance, in particular the largest distance, separating the at least one inlet from the at least one outlet from the compartment according to the X direction. Nonetheless, the invention is not limited to this embodiment and a height of the current collector smaller than 90% of the height of the microbial biofilm compartment could be considered.

Furthermore, the current collector may conform to the shape of the microbial biofilm compartment, which allows further limiting, and even suppressing, any dead volume during operation. In particular, the current collector conforms to the shape of the microbial biofilm compartment over the entire height of the current collector (measured according to the X direction), this height may be smaller than the height of the microbial biofilm compartment, as explained hereinabove. Advantageously, the current collector may thus occupy 90 to 100% of the internal volume of the microbial biofilm compartment.

The reactor typically has a parallelepipedal or cylindrical shape.

According to the invention, inside at least one chamber so-called the fluidisation chamber, the height at rest of the granular support material is adapted to a fluidisation of said granular support material. Such a height is smaller than the height of the fluidisation chamber. This height may be determined by calculation and/or experiments. Typically, this height does not exceed 75%, and possibly 50% of the height of said fluidisation chamber, these heights being measured according to the X direction. In other words, the maximum height of the granular support material may represent 75%, and possibly 50% of the total height of the fluidisation chamber. Advantageously, the minimum height at rest of the granular support will be different from zero and could be calculated so that the amount of granular support material is optimum for the formation of a biofilm. This minimum height could be determined by calculation and/or experiments. Typically, the height at rest of the granular support material inside a fluidisation chamber may extend over 10 to 75% of the height of the chamber, in particular over 10 to 50% of the height of the chamber or may be within any range defined by a combination of these limits.

Advantageously, the total height at rest of the granular support material corresponding to the sum of the heights at rest of the granular support material in each of the chambers of the current collector of the microbial biofilm compartment, could range from 10 to 75% of the total height of the current collector, in particular from 10 to 50% of the total height of the current collector or within any range defined by a combination of these limits.

In particular, it is possible to provide visual markings on the collector allowing filling each of its chambers with the granular support material at the desired height or directly introducing the corresponding volume of support material into each chamber or else converting this volume into mass (taking into account the possible difference between the apparent volumetric mass of the granules and the actual mass of the material because of the air present between the granules) and weighing the mass of the granular support material that should be introduced.

Typically, sizing of the microbial biofilm compartment and of the current collector, in particular when the latter occupies the entire volume of the compartment, could be selected according to the ratio D_{T}/dₚ with D_{T} the diameter of the fluidised bed (or equivalent diameter if the section of the current collector is not a disc) and dₚ the average diameter of the granules.

For example, it is possible to consider that a homogeneous expansion of the fluidised bed is obtained for a ratio higher than 12 in the ideal case of a cylindrical current collector, of homogeneous and spherical support material granules. Thus, the diameter D_{T} obtained for a ratio of 12 corresponds to a minimum value of the section of the microbial biofilm compartment. In a manner known to a person skilled in the art, the maximum value of this section depends on the flow rate of the effluent, the nature of the process, the stay time. Typically, this ratio could be from 10 to 15, most often from 12 to 15 in order to limit the wall effects.

Once the section of the current collector is thus determined, it is possible to select a height/section ratio of the relevant current collector according to the stay time required to treat an effluent given by a given process, the amount of granular support material, the implemented biological kinetics. The selection of the height of the current collector may also be constrained by the pressure drop and the need to fluidise the granules of the granular support material in all fluidisation chambers. In general, a person skilled in the art could determine the dimensions of the microbial biofilm compartment by calculation and/or experiments. Thus, it should be noted that the current collector has, over its entire height, a section identical to an inner section of the microbial biofilm compartment. In other words, the section of the current collector extends over the entire surface of the inner section of the microbial biofilm compartment.

Advantageously, in order to facilitate fluidisation, the height of each fluidisation chamber measured according to the X direction may be adapted for optimal fluidisation. In particular, this height may be determined according to characteristic parameters of the granular support material such as density, geometry and size of the particles of the granular support material and of at least one characteristic parameter of the circulation of the fluid (effluent) inside the microbial biofilm compartment, such as its superficial velocity. A person skilled in the art could determine this height by calculation and/or experiments.

By “biocompatible granular support material”, it should be understood a support material in granular form (which is in the form of particles or granules) and on which microorganisms can grow.

The granular support material may be an electrically-conductive material, so as to have capacitive effects, such as granular graphite, granular activated carbon (GAC), biochar or magnetite or a composite material having a conductive outer layer. Such a composite material may have a core made of a barely dense material covered with a conductive coating, which can allow obtaining a support material that is less dense than the fluid to be treated.

Alternatively, the granular support material may be an electrically non-conductive material, for example a polymer material such as polyethylene. The capacitive effect is then based on that of the formed biofilm.

By “biocompatible granular support material”, it should be understood a support material in a granular form (which is in the form of particles or granules) and on which microorganisms can grow.

Advantageously, the granular support material may be porous, so as to have a large (apparent) surface, to maximise the possibilities of attachment of the microorganisms. It is possible to use a granular support material having pores with a diameter from 1 to 100 μm, typically from 10 to 100 μm. The distribution of the size of the pores could be determined by nitrogen volumetry based on the adsorption isotherms recorded at 77 K by applying the methods well known to a person skilled in the art (Barett, E. P.; Joyner, L. G.; Halenda, P. P. J. Am. Chem. Soc. 1951, 73, 373-380). The selection of the granular support material, and in particular its pore distribution, its pore volume and/or its specific surface, is done by tests with conventional techniques known to a person skilled in the art and depends on the nature of the microorganisms of interest.

It will be preferable to use a granular support material with a grain size smaller than or equal to 2 cm, and possibly 1 cm, for example from 0.2 to 2 mm, in particular equal to 2 mm or within any range defined by a combination of these limits. The grain size distribution is the statistical distribution of the size of the granules. It may be measured by sieving or by laser diffraction.

The volumetric mass of the granular support material may be selected according to the direction of fluidisation, ascending or descending, in other words according to the difference in volumetric mass between the effluent to be treated and the support material. The effluent to be treated comprising most often 50% by volume or more of water, it is possible to consider, as the case may be, that the volumetric mass of the effluent is equal to that of water or close to that of water.

For example, the granular support material could have a volumetric mass higher than that of water, in particular higher than 1,000 kg/m³, advantageously lower than or equal to 3,000 kg/m³, for example from 1,100 kg/m³ to 2,500 kg/m³ or in any range defined by a combination of these limits. In this case, the flow of the fluid used to fluidise the support material will be ascending to fluidise the granules. Alternatively, the granular support material could have a volumetric mass lower than that of water, in particular lower than 1,000 kg/m³, for example from 100 to 900 kg/m³ or in any range defined by a combination of these limits. In this case, the flow of the fluid used to fluidise the support material will be descending to fluidise the granules.

The volumetric mass of the granular support material could be measured by weighing in a pycnometer.

The reactor, according to the invention, may further comprise means for regulating the pH, the temperature, and/or the electrolyte level, preferably, in each of the anode and cathode compartments.

The reactor according to the invention may comprise a multistack-type structure, with a (horizontal) series of an anode compartment, a first inter-membrane compartment, a cathode compartment, a second inter-membrane compartment, it being understood that one end of this series is an anode, and the other is a cathode.

The invention also relates to a method for treating a liquid effluent implementing a bio-electrochemical reactor according to the invention under conditions that allow fluidising the granular support material present in one or more chamber(s) of the current collector.

Typically, this fluidisation may be obtained by the circulation of the effluent to be treated optionally supplemented by the circulation of a fluidisation gas introduced to this end into the reactor. This fluidisation gas may be a gas that is inert with respect to the bio-electrochemical reactions in presence or participate in these reactions (for example in the case of a microbial biofilm cathode compartment of an electro-synthesis reactor into which a carbonaceous source is introduced in the form of a gas).

Thus, in the method according to the invention, the effluent to be treated is introduced into the at least one microbial biofilm compartment of the bio-electrochemical reactor, and the effluent to be treated is circulated, in particular continuously, inside the microbial biofilm compartment with a flow rate higher than or equal to a minimum fluidisation flow rate of the granular support material contained inside the current collector. The circulation of the effluent to be treated at this flow rate is carried out by the means for circulating the fluid of the bio-electrochemical reactor of the present invention.

Thus, during circulation thereof inside the at least one microbial biofilm compartment, the effluent is subjected to a step of treatment by a bio-electrochemical reaction catalysed by the electroactive microorganisms contained in the at least one microbial biofilm compartment and a treated or partially treated effluent is recovered at the outlet of the latter.

Advantageously, it is possible to provide for introducing a fluidisation gas into said at least one microbial biofilm compartment upstream of at least one fluidisation chamber, for example downstream of a chamber of the current collector, preferably a chamber located immediately proximate to the at least one inlet of the fluid to be treated inside the compartment.

Advantageously, it is possible to provide for a recycling of the treated or partially treated effluent. Thus, it is possible to return at least part of the treated or partially treated effluent coming out of said at least one microbial biofilm compartment into said compartment upstream of at least one fluidisation chamber, optionally downstream of a chamber of the current collector, preferably a chamber located immediately proximate to the at least one inlet of the fluid to be treated inside the compartment. It should be noted that it is possible to recycle all or part of the treated or partially treated effluent upstream of one or more fluidisation chamber(s), and downstream of a chamber of the collector, which then operates as a fixed-bed chamber.

It should be noted that the effluent to be treated then circulates inside the microbial biofilm compartment mixed with the treated or partially treated effluent recycle with a flow rate higher than or equal to the minimum fluidisation flow rate of the granular support material contained inside the current collector. Thus, the effluent to be treated, possibly mixed with the recycled portion of the treated or partially treated effluent, forms a continuous liquid phase which will fluidise the granular support material. An additional injection of a fluidisation gas allows enhancing the fluidisation of the granular support material.

In general, this minimum flow rate may be determined based on a minimum superficial velocity of fluidisation of the fluid used to fluidise the support material, namely the effluent to be treated, alone or mixed with the recycled portion of the treated or partially treated effluent. This minimum velocity is a minimum fluidisation velocity of all particles. In the case where these particles have different sizes, the fluidisation of the largest particles is typically desired.

By “superficial velocity”, it should be understood the hypothetical flow velocity calculated as if the given phase or fluid was the only one to flow or to be present in a given section of the microbial biofilm compartment. The superficial velocity may be defined as the ratio of the volume flow rate of the phase or of the fluid (m³/s) to the surface of the section of the reactor (m²). Thus, it is easy to access the fluid flow rate by determining the superficial velocity.

When the fluid ensuring fluidisation is the effluent to be treated, alone or mixed with all or part of the treated or partially treated effluent recycle, the minimum superficial velocity of fluidisation of the support material may be calculated according to the following formula:

$${U}_{mf} = \qquad [ Maths 1 ]$$

$$\frac{μ}{{ρ}_{f} \cdot {d}_{p}} \sqrt{{[ 3 \cdot ( 1 - {ε}_{mf} ) \cdot \frac{{h}_{K}}{{h}_{B}} ]}^{2} + \frac{{ε}_{mf}^{3}}{6 \cdot {h}_{B}} \cdot \frac{{d}_{p}^{3} \cdot g \cdot ( {ρ}_{p} - {ρ}_{f} ) \cdot {ρ}_{f}}{{μ}^{2}}} -$$

$$3 \cdot ( 1 - {ε}_{mf} ) \cdot \frac{{h}_{K}}{{h}_{B}}$$

With:
U_{mf} the minimum fluidisation velocity
μ the viscosity of the fluid (effluent to be treated, alone or mixed with the recycle)
ρᵣ and ρₚ the volumetric masses of the fluid and of the support material respectively
dₚ the diameter of the granules of the support material
ε_{f} the porosity of the bed of granules of the support material (0.4 for spherical granules)
g the acceleration of gravity
h_{K}=4.14 and h_{B}=0.29 the constants of Ergün's law

Other empirical relationships may be used. Based on the minimum superficial velocity of fluidisation and knowing the section of the reactor, the minimum effluent flow rate necessary for the fluidisation of the support material is deduced.

When the fluid ensuring fluidisation is the effluent to be treated, alone or mixed with all or part of the treated or partially treated effluent recycle, mixed with a fluidisation gas, the minimum superficial velocity of fluidisation of the support material may be determined based on correlations integrating the rates of injection of the fluidisation gas, such as co-current (Larachi et al., 2000, Ind. Eng. Chem. Res., 39, 563-572) or counter-current (Sur et al., 2017, Journal of Environmental Chemical Engineering, 5, 3518-3528) tri-phase correlations.

Moreover, advantageously, the superficial velocity of circulation of the fluid ensuring fluidisation, and consequently its volume flow rate, will be lower than an entrainment velocity of the particles corresponding to the velocity from which the smaller particles are entrained.

In the presence of a fluidisation gas, the superficial velocity of circulation of the fluid ensuring fluidisation, and consequently its volume flow rate, may be lower than a velocity so-called “bubbling velocity” corresponding to the velocity from which the fluidised bed is no longer homogeneous and where effluent “bubbles” or pockets will form in the granular bed. There are empirical formulas for estimating this velocity. For granules with a large diameter, it is unlikely that this velocity is reached.

In general, the direction of circulation of the fluid ensuring fluidisation, namely the effluent to be treated alone or mixed with recycling, will be selected according to the volumetric mass of the support material.

Thus, the direction X of circulation of the effluent to be treated inside the anode compartment being parallel to the direction of gravity, the effluent to be treated, alone or mixed with the recycled portion of the treated or partially treated effluent, circulates:
according to an ascending current when the density of the granular support material is higher than its density, or
according to a descending current when the density of the granular support material is lower than its density.

When a fluidisation gas is used to fluidise the support material, it could indifferently circulate in a co-current or counter-current manner with the effluent to be treated, the latter case could for example be selected when the density of the granular support material is lower than the density of the effluent to be treated.

Typically, the stay time of the effluent inside the microbial biofilm compartment is from 1 to 48 hours, for example 6 h or within any range defined by a combination of these values.

In particular, the use of recycling allows adjusting the stay time of the effluent to be treated to the pollution content to be treated, in particular according to the desired COD reduction rate (in the case of an anode microbial biofilm compartment).

Advantageously, different electroactive microorganisms may be introduced into at least two distinct chambers of the current collector of the microbial biofilm compartment.

Alternatively or in combination, it is also possible to select, in at least two distinct chambers of the current collector of the microbial biofilm compartment, a specific granular support material and/or a specific superficial velocity of the effluent to be treated (for example to form a fixed or fluidised bed), which promotes the development of microorganisms catalysing a specific electrochemical reaction. This allows implementing different microbial ecology strategies enabling a differentiated selection of the microorganisms in distinct chambers of the current collector.

For example, when the current collector of the microbial biofilm compartment of the reactor is an anode compartment, it may have, from upstream to downstream according to the direction of circulation of the effluent:
at least one stage in which the dioxygen contained in the effluent is used to oxidise the COD contained therein,
one or more downstream stage(s) operating in anaerobiosis.

According to another example, when the current collector of the microbial biofilm compartment of the reactor is a cathode compartment (case of a microbial fuel cell), it may have, from upstream to downstream according to the direction of circulation of the effluent:
at least one stage for reducing O₂,
at least one other stage ensuring the denitrification of the effluent to be treated.

According to still another example, when the current collector of the microbial biofilm compartment of the reactor is a cathode compartment (case of an electrosynthesis reaction), it may have, from upstream to downstream according to the direction of circulation of the effluent of the stages corresponding to the different steps of synthesis of a complex molecule like, for example, the synthesis of caproic acid. It is then possible to provide for a stage for the reduction of CO₂ into acetate, a stage for the reduction of acetate into ethanol and a stage for the production of caproic acid by reduction of ethanol, acetate and CO₂.

Advantageously, as already explained, it is possible to use different granular support materials in at least two distinct chambers of the current collector of the microbial biofilm compartment, in combination or not with the introduction of different electroactive microorganisms and/or with the selection of a specific granular support material and/or of a specific superficial velocity of the effluent to be treated.

The reactor and the method according to the present invention are useful for the treatment of effluents comprising a biodegradable organic matter, such as a wastewater effluent or an organic waste hydrolysate. In particular, it may consist of sludge or wastewater from a wastewater treatment plant.

The effluents comprising a biodegradable organic matter used in the invention are typically: manure, leachate, bio-waste hydrolysates, hydrolysed sludge from wastewater treatment plants, different organic liquid fractions of wastewater treatment plants, urban wastewater after primary settling, organic industrial effluents, for example derived from agrifood industries, digestates of wastewater treatment plants, or a mixture of several of these effluents.

These effluents typically contain more than 50% by volume of water, in general more than 60% by volume of water. In some cases, the water content may be at least 95% by volume, or even at least 99% by volume, for example up to 99.9% by volume. The water content may be in any range defined by the aforementioned limits. In general, the remaining percentages are solids, such as particles, suspended materials, colloids, etc. Preferably, the effluent is a wastewater effluent, for example organic liquid fractions of wastewater treatment plants, urban wastewater, in particular after primary settling, organic industrial effluents (for example derived from agrifood industries), or a mixture thereof.

Thus, the electrolyte of the microbial biofilm compartment contains such organic carbon effluents in the liquid form, introduced either raw or diluted in a synthetic base electrolyte. In this microbial biofilm compartment, the content of organic matters quantified by the measurement of the COD is advantageously comprised between 0.01 and 200 g/L, preferably between 0.1 and 20 g/L, still more preferably between 0.5 and 5 g/L.

The COD is the measurement of all oxidisable substances, whether these are biodegradable or not. The COD may be measured according to the standard NFT 90-101-February 2001 or ISO 6060-1989.

The reactor and the method according to the invention may also be used to carry out a denitrification treatment of an effluent of the aforementioned type.

The reactor and the method according to the invention may be used for the production by electrosynthesis of organic waste, and in particular by electrosynthesis of one or more of the abovementioned effluents, of dihydrogen or of organic molecules of interest selected from among organic acids, alcohols, methane.

The reactor and the method according to the invention enable a continuous treatment of an effluent to be treated, suitable for an industrial application.

### DESCRIPTION OF THE FIGURES

The invention is now described with reference to the non-limiting appended drawings, wherein:

FIG. 1 schematically shows an embodiment of a bio-electrochemical reactor according to the invention comprising a microbial biofilm compartment.

FIG. 2 schematically shows another embodiment of a microbial biofilm compartment of a reactor according to the invention.

FIG. 3 schematically shows another embodiment of a microbial biofilm compartment of a reactor according to the invention.

FIGS. 4A and 4B schematically show a first case of circulation of the fluids throughout a current collector, respectively without and with a fluidisation gas.

FIGS. 5A and 5B schematically show a second case of circulation of the fluids throughout a current collector, respectively without and with a fluidisation gas.

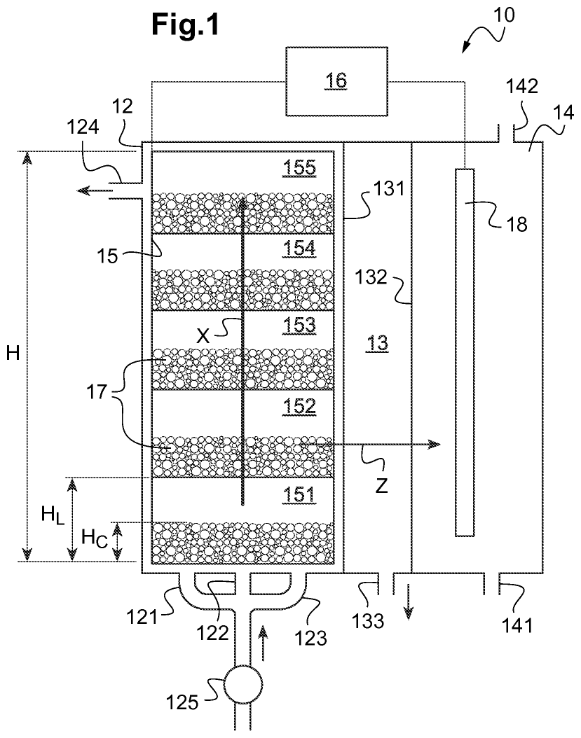

FIG. 1 shows a bio-electrochemical reactor 10 including an anode compartment 12 and a cathode compartment 14 separated by a separator 13.

In the illustrated example, the anode compartment 12 is a microbial biofilm compartment as defined in the present invention. Thus, it comprises three inlets 121, 122, 123 for the effluent to be treated, herein located at a lower end of the compartment 12 and an outlet 124 of the treated effluent located at an upper end of the compartment 12. Means 125 for circulating the fluid inside the compartment, such as a pump, or other, enable the circulation of the fluid according to an X direction between the inlets 121-123 and the outlet 124. The X direction is herein a vertical ascending direction, perpendicular to the Z direction, herein horizontal, extending from the anode compartment 12 to the cathode compartment 14. Of course, the invention is not limited by the number of inlets and/or outlets of the effluent, nor by the nature of the circulation means provided that the effluent to be treated could circulate inside the compartment 12.

The compartment 12 further comprises a multi-stage current collector 15 electrically connected to a device 16 which may be an electrical component (for example an electrical resistor) for use of the reactor as a fuel cell or a device for applying a voltage for use of the reactor as an electrolysis reactor or an electrosynthesis reactor.

The illustrated multi-stage current collector 15 comprises 5 stages each defining a chamber 151-155 containing a granular support material 17. For example, the multi-stage current collector 15 is formed from a mesh made of a conductive material, for example made of stainless steel. Advantageously, the collector 15 is removable to facilitate filling of the chambers. To this end, it is possible to provide for an opening for filling each chamber which may be closed by a door or make a modular structure wherein each chamber defines a removable container, for example in the form of a basket, which may be inserted/extracted from a support structure, the entirety of the support structure and the removable containers being made of a conductive material and electrically connected to form the current collector. Of course, the invention is not limited by the shape of the current collector, provided that the latter is an electrical conductor and that it lets the effluent to be treated pass while retaining the granular support material 17.

Each chamber 151-155 has a height H_{C} and receives the granular support material 17 over a height H_{L}, at rest, i.e. in the absence of circulation of fluid inside the compartment. These heights are measured parallel to the X direction. This height H_{L}, at rest, corresponds for example to half the height H_{C} to facilitate the fluidisation of the support material inside the chambers.

In the illustrated example, the current collector 15 extends over the entire height H of the compartment 12 wherein it conforms to the shape thereof: its inner volume is therefore substantially identical to the inner volume of the compartment 12, thereby limiting dead volumes. In the example, the chambers 151-155 have substantially the same height, however, the invention is not limited to particular dimensions of the chambers, which may have different dimensions, in particular to receive different heights of support material. Nonetheless, as shown in the figures, each chamber 151-155 extends over the entire surface of the inner section of the compartment 12. The current collector 15 thus conforms, over its entire height according to the X direction, to the shape of the inner volume of the compartment 12. Thus, all of the treated effluent passes through each chamber.

In the illustrated example, the cathode compartment 14 includes an electrode 18 immersed in an electrolyte circulating inside the compartment between an inlet 141 and an outlet 142, the electrode 18 being electrically connected to the device 16. Furthermore, the separator 13 is herein in the form of an inter-membrane compartment defined by ion-exchange walls 131 and 132. Typically, one of the membranes 131, 132 is a cation-exchange membrane and the other one is an anion-exchange membrane. A drawing device comprising, for example, an outlet 133 connected to a pump or other (not shown) may be provided as shown.

The invention is not limited by the nature of the cathode compartment, which may be a bio-cathode comprising an electrolyte containing electroactive microorganisms having a structure identical to that of the anode compartment or a structure similar to the existing ones (fixed-bed, brush, plate type granular electrode, etc.).

The invention is neither limited by the shape of the separator 13 provided that the latter is permeable to the ions that are intended to circulate between the cathode and the anode.

In the case where the granular support material 17 is denser than the effluent, during operation of the reactor 10, the circulation of the effluent throughout the stages of the current collector 15 according to the ascending X direction allows fluidising the granular material of each of the chambers 151-155: the latter will thus be distributed in a relatively homogeneous manner inside each chamber, herein over the entire height of the compartment 12, enabling the development of a biofilm over most granules of the support material 17. Furthermore, because of the stirring of the granules inside each chamber due to fluidisation, these granules regularly come into contact with the current collector, thereby allowing discharging it. Thus, it should be understood that the active surface of the bio-electrode is high and that the recovery of the charges carried by the granules could be improved because of the relatively high frequency of contact of the granules with the current collector.

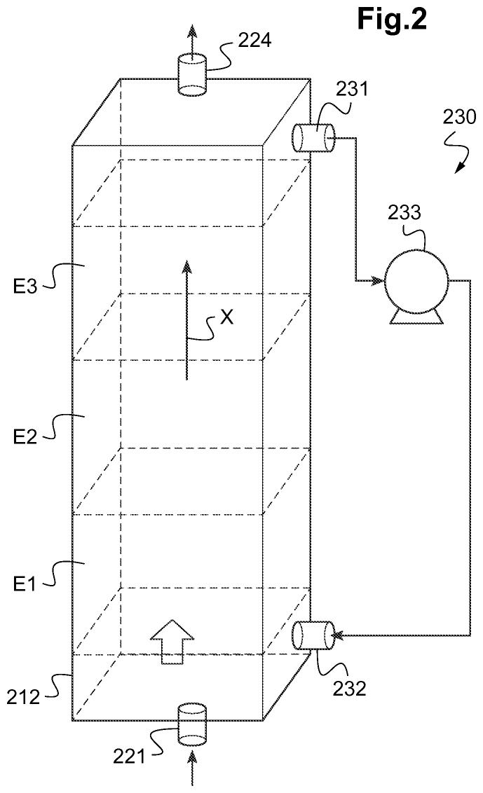

FIG. 2 shows a microbial biofilm compartment 212 similar to that described with reference to FIG. 1 but comprising a recycle circuit. The compartment 212 comprises a multi-stage current collector herein comprising three stages E1 to E3 each defining a chamber intended to receive the support material (not shown). As shown in this figure, the section of each chamber extends over the entire surface of the inner section of the compartment 212.

The effluent enters the compartment 212 through an inlet 221 located at its lower end. The treated effluent comes out via an outlet 224 located at the upper end of the compartment 212. A recycling circuit 230 is further provided between a second outlet 231 also located on the side of the upper end of the compartment 212 and a second inlet 232 located upstream of the current collector, in other words upstream of the first stage E1 with respect to the circulation of the effluent inside the compartment, herein according to an ascending current (cf. the X direction in the figures). A circulation means 233 (pump, etc.) ensures the circulation of the treated effluent in the recycling circuit 230. Thus, in operation, all or part of the effluent circulating inside the compartment 212 is recycled via the recycling circuit upstream of the first chamber of the current collector (the closest chamber to the inlet of the effluent to be treated), enabling the fluidisation of the support material at each of the stages E1-E3.

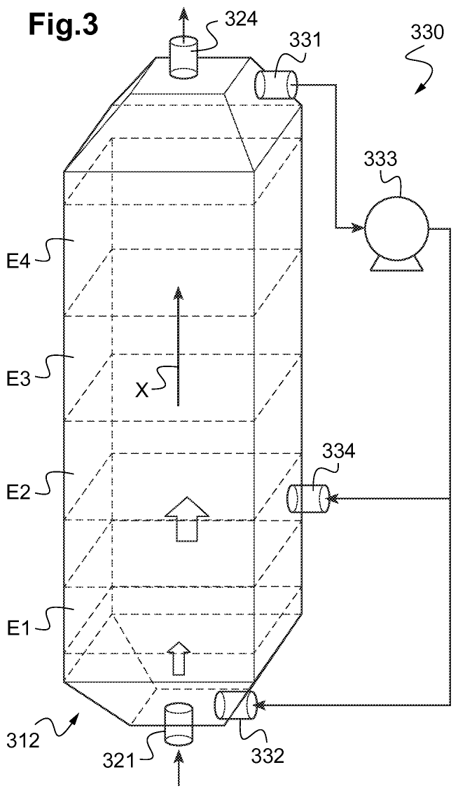

FIG. 3 shows a microbial biofilm compartment equipped with another recycling system. The compartment 312 comprises a multi-stage current collector herein comprising four stages E1 to E4 each defining a chamber intended to receive the support material (not shown). As shown in this figure, the section of each chamber extends over the entire surface of the inner section of the compartment 312.

The effluent enters the compartment 312 through an inlet 321 located at its lower end. The effluent thus also circulates according to an ascending current inside the compartment (see the X direction in the figures). The treated effluent comes out through an outlet 324 located at the upper end of the compartment 312. A recycling circuit 330 connects a second outlet 331 also located on the side of the upper end of the compartment 312 and a second inlet 334 located between the first stage E1 and the second stage E2 of the current collector. A circulation means 333 (pump, etc.) ensures the circulation of the treated effluent in the recycling circuit 330. Thus, in operation, all or part of the effluent circulating inside the compartment 312 is recycled via the recycling circuit downstream of the first chamber of the current collector (the closest chamber to the inlet of the effluent to treating), enabling the fluidisation of the support material at each of the stages located downstream, namely the stages E2 to E4, while the support material of the first stage operates as a fixed bed. The recycle circuit 330 may also be connected to another inlet 332 located upstream of the first stage E1 so that depending on needs, the recycle could enable the fluidisation of the support material at all stages, as in the embodiment described with reference to FIG. 2.

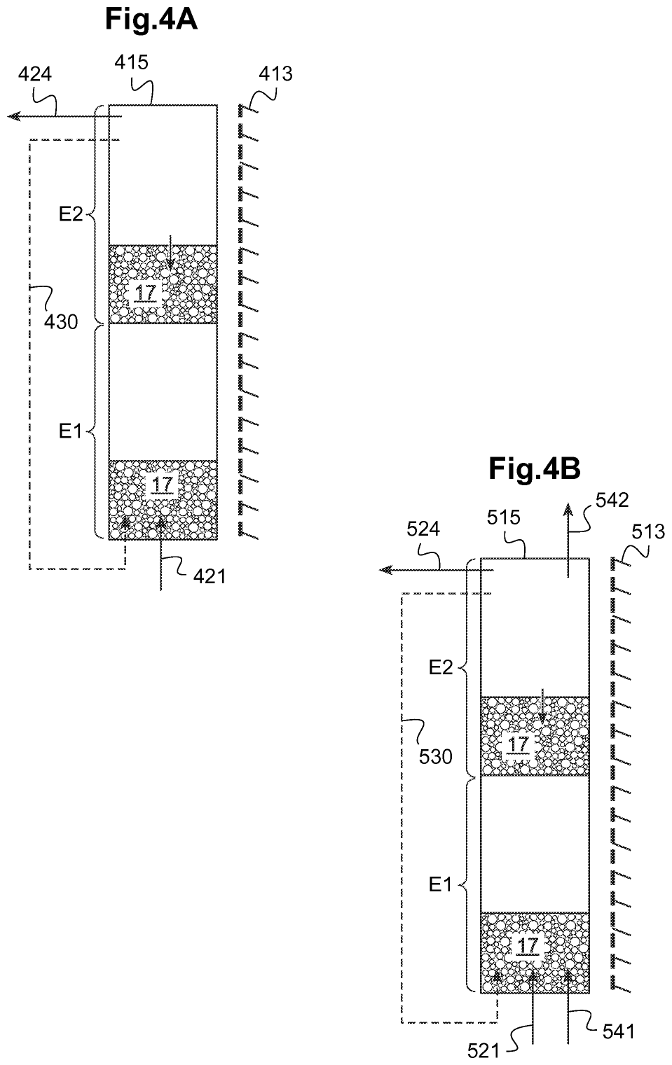

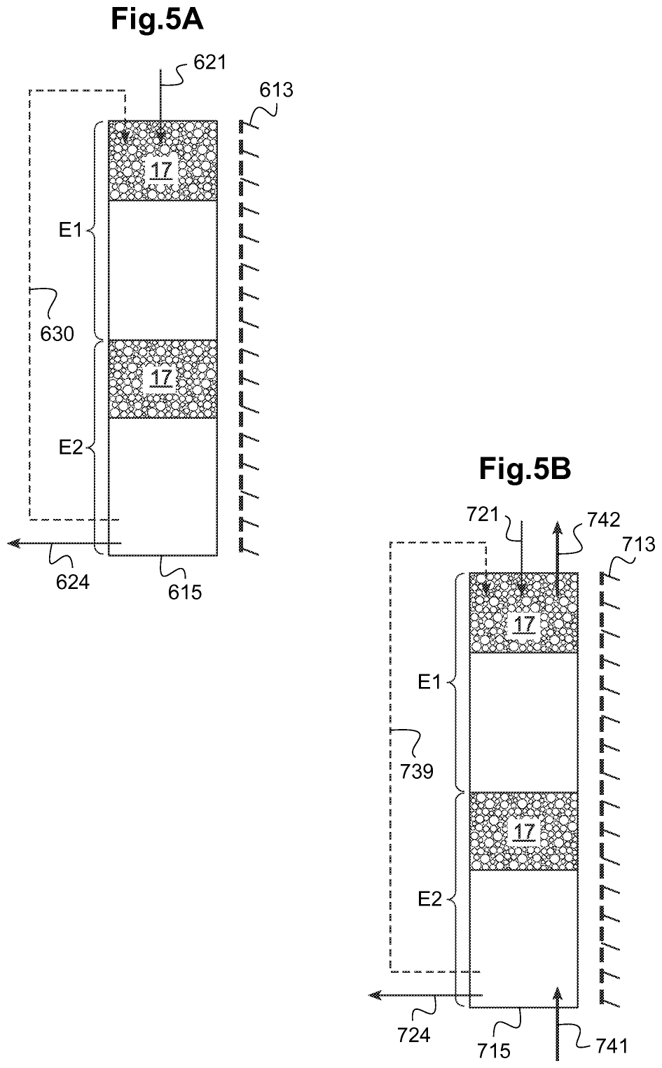

In the examples shown in FIGS. 1 to 3, the fluidisation of the support material is obtained by the circulation of the effluent throughout the chambers of the current collector, this fluidisation may also be promoted by injection of a fluidisation gas. FIGS. 4A, 4B, 5A, 5B schematically illustrate the possible circulations of the fluids inside microbial biofilm compartments according to the invention according to different cases. These different circulation configurations may be implemented regardless of the arrangement of a microbial biofilm compartment as defined in the present invention. Each of these figures schematically shows a multi-stage current collector 415, 515, 615, 715 with two stages E1, E2, as well as the fluid circulations throughout this current collector. Each stage includes a chamber containing support material 17. As shown in these figures, the section of each chamber extends over the entire surface of the inner section of the microbial biofilm compartment.

The case shown in FIG. 4A corresponds to a case with no fluidisation gas, wherein the density of the support material 17 is higher than the density of the effluent. The effluent then circulates throughout the current collector 415 according to an ascending current between an inlet 421 of the compartment and an outlet 424. A recycling circuit 430 similar to that described with reference to FIG. 2 may be provided, or not.

The case shown in FIG. 4B corresponds to a case with a fluidisation gas, wherein the density of the support material 17 is higher than the apparent density of the effluent-fluidisation gas mixture. The effluent then also circulates throughout the current collector 515 according to an ascending current between an inlet 521 of the compartment and an outlet 524, as well as the fluidisation gas which circulates between a lower inlet 541 and an upper outlet 542. A recycling circuit 530 similar to that described with reference to FIG. 2 may be provided, or not.

The case shown in FIG. 5A corresponds to a case with no fluidisation gas, wherein the density of the support material 17 is lower than the density of the effluent. The effluent then circulates throughout the current collector 615 according to a descending current between an upper inlet 621 of the compartment and a lower outlet 624. A recycling circuit 630 may be provided as shown, or not.

The case shown in FIG. 5B corresponds to a case with a fluidisation gas, wherein the density of the support material 17 is lower than the apparent density of the effluent-fluidisation gas mixture. The effluent then also circulates throughout the current collector 715 according to a descending current between an upper inlet 721 of the compartment and a lower outlet 724. In contrast, the fluidisation gas circulates according to an ascending current between a lower inlet 741 and an upper outlet 742. A recycling circuit 730 may be provided, or not.

### REFERENCES

[1] Deeke, A., T. H. J. A. Sleutels, T. F. W. Donkers, H. V. M. Hamelers, C. J. N. Buisman, et A. Ter Heijne. 2015. “Fluidised capacitive bioanode as a novel reactor concept for the microbial fuel cell”. Environmental Science and Technology 49(3):1929-35
[2] Jia, Y. H., J. H. Ryu, C. H. Kim, W. K. Lee, T. V. T. Tran, H. L. Lee, R. H. Zhang, et D. H. Ahn. 2012. “Enhancing hydrogen production efficiency in microbial electrolysis cell with membrane electrode assembly cathode”. Journal of Industrial and Engineering Chemistry 18(2):715 19
[3] Li, J., Z. Ge, et Z. He. 2014. “A fluidised bed membrane bioelectrochemical reactor for energy-efficient wastewater treatment”. Bioresource Technology 167:310 15.
[4] Liu, J., F. Zhang, W. He, W. Yang, Y. Feng, et B. E. Logan. 2014. “A microbial fluidised electrode electrolysis cell (MFEEC) for enhanced hydrogen production”. Journal of Power Sources 271:530 33.
[5] Liu, J., F. Zhang, W. He, X. Zhang, Y. Feng, et B. E. Logan. 2014b. “Intermittent contact of fluidised anode particles containing exoelectrogenic biofilms for continuous power generation in microbial fuel cells”. Journal of Power Sources 261:278 84.
[6] Zhou, Y., G. Zhou, L. Yin, J. Guo, X. Wan, et H. Shi. 2017. “High-Performance Carbon Anode Derived from Sugarcane for Packed Microbial Fuel Cells”. ChemElectroChem 4(1):168-74

1. A bio-electrochemical reactor for treating a liquid effluent, comprising at least one anode compartment and at least one cathode compartment, at least one separator located between the at least one anode compartment and the at least one cathode compartment, characterised in that at least one amongst the anode and cathode compartments is a microbial biofilm compartment including:
at least one fluid inlet located at one end of the compartment,
at least one fluid outlet located at an opposite end of the compartment,
means for circulating the fluid inside the compartment between the at least one inlet and the at least one outlet according to an X direction,
an electrolyte comprising electroactive microorganisms,
a biocompatible granular support material,
a multi-stage current collector located between the at least one fluid inlet and the at least one fluid outlet of said microbial biofilm compartment, said collector comprising at least two stages each defining a chamber serving as a container for the granular support material and letting the fluid pass, said collector conforming to the shape of the microbial biofilm compartment over the height of said collector measured according to the X direction and having a structure provided with a multitude of orifices not letting the particulate material pass,
and in that,
the direction X of circulation of the fluid inside said microbial biofilm compartment is not parallel to a direction Z from the anode compartment to the cathode compartment, and,
inside at least one chamber of the current collector so-called the fluidisation chamber, a height at rest of the granular support material measured according to the X direction in the absence of fluid circulation inside the microbial biofilm compartment, is a height suitable for fluidisation of said granular support material smaller than the total height of the fluidisation chamber.

2. The bio-electrochemical reactor according to claim 1, characterised in that the anode compartment is a microbial biofilm compartment, and, optionally, the cathode compartment is a microbial biofilm compartment.

3. The bio-electrochemical reactor according to claim 1, characterised in that said reactor is an electrolysis reactor or a microbial electrosynthesis reactor comprising means for applying a potential difference between the current collector of the microbial biofilm compartment and the electrode of the other compartment.

4. The bio-electrochemical reactor according to claim 1, characterised in that the separator comprises a cation-exchange membrane and an anion-exchange membrane separated from each other by an inter-membrane compartment comprising a device for drawing molecules synthesised within said reactor, the membranes being optionally positioned so that the anode compartment is separated from the cathode compartment, from the anode compartment to the cathode compartment, by said cation-exchange membrane and said anion-exchange membrane.

5. The bio-electrochemical reactor according to claim 1, characterised in that the at least one microbial biofilm compartment includes at least one recycling circuit connecting the at least one outlet to the at least one fluid inlet or connecting the at least one outlet to at least one recycling inlet opening into one of the chambers or upstream of one of the chambers with respect to the circulation of the fluid.

6. The bio-electrochemical reactor according to claim 1, characterised in that it includes at least one of the following features:
the current collector has a structure having a multitude of orifices which do not let the particulate material pass through, formed by a perforated plate, a fabric or a mesh,
the current collector extends over 90 to 100% of the height of the microbial biofilm compartment, this height of the microbial biofilm compartment being defined as the distance separating the at least one inlet of the at least one outlet of the compartment according to the X direction.

7. The bio-electrochemical reactor according to claim 1, characterised in that the height of each fluidisation chamber according to the X direction is determined according to characteristic parameters of the granular support material selected from among the density, the geometry and the particle size of the granular support material and according to at least one characteristic parameter of the circulation of the effluent inside the microbial biofilm compartment, such as its superficial velocity.

8. The bio-electrochemical reactor according to claim 1, characterised in that the granular support material is selected from among a polymer material, granular graphite, granular activated carbon, biochar, magnetite, a composite material having a conductive outer layer.

9. The bio-electrochemical reactor according to claim 1, characterised in that the granular support material has a particle size smaller than or equal to 2 cm.

10. A method for treating a liquid effluent implementing a bio-electrochemical reactor according to claim 1, wherein:
the effluent to be treated is introduced into the at least one microbial biofilm compartment of the bio-electrochemical reactor and the effluent to be treated is circulated inside the microbial biofilm compartment with a flow rate higher than or equal to a minimum fluidisation flow rate of the granular support material contained inside the current collector.

11. The method for treating a liquid effluent according to claim 10, wherein at least one portion of the effluent coming out of said at least one microbial biofilm compartment is returned into said compartment upstream of at least one fluidisation chamber, optionally downstream of a chamber of the current collector.

12. The method for treating a liquid effluent according to claim 10, wherein the direction X of circulation of the effluent to be treated inside the anode compartment is parallel to the direction of gravity and the effluent to be treated, alone or mixed with the recycled portion of the effluent, circulates
according to an ascending current when the density of the granular support material is higher than its density, or
according to a descending current when the density of the granular support material is lower than its density.

13. The method for treating a liquid effluent according to claim 10, comprising, in at least two distinct chambers of the current collector of the microbial biofilm compartment, at least one of the following features:
different electroactive microorganisms are introduced,
a specific granular support material and/or a specific superficial velocity of the effluent to be treated is selected which promotes the development of microorganisms catalysing one specific electrochemical reaction,
different granular support materials are used.

14. The treatment method according to claim 10, wherein the effluent to be treated is selected from among manure, leachate, bio-waste hydrolysates, hydrolysed sludge from wastewater treatment plants, different organic liquid fractions of wastewater treatment plants, urban wastewater after primary settling, organic industrial effluents, agrifood industries effluents, digestates of wastewater treatment plants, or a mixture of several thereof.

15. A use of the reactor according to claim 1 to produce dihydrogen or organic molecules of interest selected from among organic acids, alcohols, methane, by electrosynthesis of organic waste.
